(19) United States
(12) Patent Application Publication
(10) Pub. No.: US 2010/0015619 A1
(43) Pub. Date: Jan. 21, 2010

(54) METHOD OF DETECTING GENOMIC ABERRATIONS FOR PRENATAL DIAGNOSIS

(75) Inventors: Hongtao Zhang, Beijing (CN); Ning Liu, Beijing (CN); Zhong Chen, Sandy, UT (US)

Correspondence Address: WEILI CHENG, CLAYTON, HOWARTH & CANNON, P.C., P.O.BOX 1909, SANDY, UT, 84091, US

(73) Assignee: CYTOTREND (BEIJING) BIOTECH ENGINEERING CO., LTD., Beijing (CN)

(21) Appl. No.: 12/445,516

(22) PCT Filed: Sep. 21, 2007
(86) PCT No.: PCT/US07/79218

§ 371 Date: Apr. 14, 2009

Related U.S. Application Data
(60) Provisional application No. 60/863,439, filed on Oct. 30, 2006.

(51) Int. Cl. C12Q 1/68

(52) U.S. Cl. 435/6

(57) ABSTRACT

This invention relates to assays used to detect and confirm genomic aberrations, such as chromosomes 13, 18, 21, X and Y aneuploidy as well as 22q11.2 deletions, for prenatal diagnosis. For the detection, combined STR markers (all tetra-nucleotide repeats) are employed to cover different chromosome regions. For the confirmation step, individual chromosome specific STR markers (tetra-nucleotide repeats) are utilized. This invention particularly relates to multiplex analysis for the presence or absence of STR markers in genomic DNA isolated from peripheral blood, amniotic fluid, cultured amniocytes, chorionic villi, or fetal cells existing in maternal blood. This invention offers an efficient approach to identify chromosomal abnormalities by using STR markers.

### CROSS-REFERENCE TO RELATED APPLICATIONS

This invention claims priority, under 35 U.S.C. § 120, to the U.S. Provisional Patent Application No. 60/863,439 filed on Oct. 30, 2006, which is incorporated by reference herein.

### TECHNICAL FIELD

The present invention relates to a diagnostic method for the detection of chromosomal abnormalities in a developing fetus and/or a new-born individual, or subsequently during adult growth. The method is based upon analysis of samples using the quantitative-fluorescent polymerase chain reaction (QF-PCR) to quantify sample DNA.

### BACKGROUND

Chromosomes 13, 18, 21, X and Y related anomalies have been observed in ⅔ of all prenatally significant chromosomal abnormalities and in 85-90% of all significant chromosomal changes at birth. Most of these abnormalities are trisomies that are the well-recognized causes of recurrent abortions, early neonatal death, congenital malformations, and developmental delay. 22q11.2 deletion may be one of the most common chromosomal disorders in human beings with the incidences ranging from 1/4,000 to 1/10,000 in live births. 22q11.2 deletion-related syndromes are commonly seen in patients with congenital heart defects (74%), particularly conotruncal malformations (e.g., tetralogy of Fallot, interrupted aortic arch, ventricular septal defect, and truncus arteriosus); palatal abnormalities (69%), particularly velopharyngeal incompetence (VPI), submucosal cleft palate, and cleft palate; characteristic facial features (present in the majority of Caucasian individuals); learning difficulties (70-90%); immune deficiency (77%); and hypocalcemia (50%). 22q11.2 deletion-related disorders commonly include DiGeorge syndrome, velocardiofacial syndrome and conotruncal anomaly face syndrome. 22q11.2 deletion syndromes are inherited in an autosomal dominant manner. About 93% of probands have a de novo deletion and 7% have inherited the deletion from a parent. Thus, the above chromosomal anomalies, i.e. chromosomes 13, 18, 21, X and Y aneuploidy as well as 22q11.2 deletion, consist of the common genomic markers for prenatal diagnosis.

Over the past 30 years, prenatal diagnosis has become a regular approach to identify genetic disorders. Fetal cells for prenatal diagnosis are normally obtained by either amniocentesis (usually at about 15 weeks of gestation) or chorionic villus sampling (CVS) (at about 9-11 weeks of gestation). Traditionally, chromosomal abnormalities may be identified prenatally by cytogenetic and/or fluorescence in situ hybridization (FISH) techniques. A basic cytogenetic analysis consists of growing cells, arresting cell division with colcemid, treatment with a hypotonic solution to swell the cells followed by fixing the chromosomes and remaining nuclei. Slides are prepared, stained using various banding methods, and examined under a light microscope. However, traditional cytogenetics is very labor intensive and requires a skilled analytic process and takes a long time to complete a study. Therefore, more convenient, practical, and efficient methods are demanded to replace or supplement traditional cytogenetics for prenatal diagnosis.

Molecular cytogenetics has expanded the field of cytogenetics. It capitalizes on the accuracy and detail of molecular studies, combined with the well-established techniques of cytogenetics, to gain a deeper understanding of chromosome structure and DNA rearrangements. DNA probes for specific loci or genes on the chromosomes are used in FISH. FISH is a technique that allows DNA sequences to be detected on metaphase chromosomes and interphase nuclei by using DNA probes specific for entire chromosomes or single unique sequences/genes. In general, a specimen is treated with heat and formamide to denature the double-stranded DNA to become single stranded. The target DNA is then available for binding to a DNA probe with a complementary sequence that is also similarly denatured and single stranded. The probe and target DNA then hybridize to each other in a duplex based on complementary base pairing. The probe DNA is labeled directly or indirectly with a fluorescent dye. Hybridization signals on a target material can be visualized through the use of a fluorescence microscope. In comparison with traditional cytogenetics, FISH helps confirm structural chromosome changes and identify markers, and offers the capability of evaluating the chromosome complement in interphase or non-dividing cells; FISH is quicker and easier to perform and requires less training for technical personnel involved in comparison with traditional cytogenetics. However, FISH is still cumbersome, labor-intensive, and expensive for prenatal diagnosis.

Recently, the introduction of molecular genetic method, such as quantitative-fluorescent polymerase chain reaction (QF-PCR), appears to be very promising for prenatal diagnosis. This type of approach does not require use of cell cultures and difficult analytic processes, and offers a very short turn-around-time (generally within 24 hours). In addition, less sample amounts may be required to complete a study as compared to FISH analysis.

QF-PCR is the most common molecular technique used for the detection of gene/chromosome copy numbers. QF-PCR involves amplification of chromosome-specific, repetitive DNA sequences, known as short tandem repeats (STRs). STRs are stable and polymorphic, primarily including tri, tetra-, or penta-nucleotides. In practice, DNA is amplified by PCR using fluorescent primers, and subsequently the amplified DNA can be visualized and quantified as peak areas of the respective repeat lengths by using an automated DNA sequencer with the Gen-Scan software. DNA amplified from normal subjects who are heterozygous (having alleles of different lengths) is expected to show two peaks with the same area. DNA amplified from subjects who are trisomic will exhibit either an extra peak (being triallelic), or only two peaks (being diallelic) with one of them being twice as large as the other.

The present invention demonstrates that QF-PCR technology can be used as a powerful tool for prenatal diagnosis in detecting common genomic aberrations, such as chromosomes 13, 18, 21, X and Y aneuploidy as well as 22q11.2 deletions.

### REFERENCES

Ochshom Y, Bar-Shira A, Jonish A, Yaron Y Rapdi prenatal diagnosis of aneuploidy for chromosomes 21, 18, 13, and X by quantitative fluorescence polymerase chain reaction. Fetal Diagn Ther 2006 21:326-331.
Cirigliano V, Lewin P, Szpiro-Tapies S, Fuster C and Adinolfi M Assessment of new markers for the rapid detection of aneuploidies by quantitative fluorescent PCR (QF-PCR) Annals of Human Genetics 2001 65:421-427
Lubin M B, Elashoff J D, Wang, S J, Rotter J I and Toyoda H Precise gene dosage determination by polymerase chain reaction; theory, methodology, and statistical approach Molecular and Cellular probes 1991 5:307-317
Diego-Alvarez D, Garcia-Hoyos, M, Trujillo M J, Gonzalez-Gonzalez C, Alba, M R, Ayuso C, Ramos-Corrales C, an Lorda-Sanchez I. Application of quantitative fluorescent PCR with short tandem repeat markers to the study of aneuplodies in spontaneous miscarriage Hum Rep 2005 20:1235-1243
Chen C P, Chern S R, and Wang W Rapid determination of zygosity and common aneuploidies from amniotic fluid cells using quantitative fluorescent polymerase chain reaction following genetic amniocentesis in multiple pregnancies. Human Reproduction 2000 15:929-939.
Grimsgaw G M, Szczepura A, Hilten M, MacDonald F, Nevin N C, Sutton F and Dhanjal Evaluation of molecular tests for prenatal diagnosis for chromosomes abnormalities Health Technology Assessment 2003 7: 1-56
Levett L J, Liddle S and Meredith R A large-scale evaluation of amnio-PCR for the rapid prenatal diagnosis of fetal trosimy Ultrasound in Obstetrics and Gynecology 2001 17:115-118.McDonald-McGinn D M, Tonnesen M K, Laufer-Cahana A, Finucane B, Driscoll D A, Emanuel B S, Zackai E H. Phenotype of the 22q11.2 deletion in individuals identified through an affected relative. Genet Med. 2001 3:23-9

### DISCLOSURE OF THE INVENTION

The rational for application of PCR techniques allows for the detection of chromosomal abnormalities is as follows. When primers flanking polymorphic STRs are employed to detect aneuploidies, normal individuals may have either two STR allelic products with a quantitative ratio of 1:1, or could be homozygous with two alleles of the same size. Samples from trisomic patients will either show three different alleles with quantitative dose ratios of 1:1:1 (trisomic tri-allelic), or two PCR products with a ratio of 2:1 (trisomic di-allelic) (Mansfield, E. S. Hum. Mol. Genet. 2 43-50 (1993); Pertl et al Lancet 343 1197-1198 (1994)). Therefore, it is possible for a trisomic sample to have three similarly sized STR alleles and so represent a single PCR product indistinguishable from a homozygote normal individual. In such circumstances, it is almost impossible to make an accurate diagnosis. Efforts to avoid this problem have included the use of a non-polymorphic marker as a control, or several STR markers for each chromosome (Pertl et al Hum. Genet 98 55-59 (1996); Pertl et al Am. J. Obs. Gyn. 177 899-906 (1997)).

However, there are problems with the currently existing methods in terms of reliability and overall accuracy. For example, errors can be introduced through sample contamination in a PCR procedure. Perhaps, the most significant cause of error is allele drop-out (ADO), or the preferential amplification of one allele (Ray et al J. Assist. Reprod. Genet. 13 (2) 104-106 (1996)). This phenomenon can lead to the distortion of the ratio of PCR product obtained. Since the medical decisions made as a result of a positive prenatal diagnosis of aneuploidy have very serious implications, there is a need to provide diagnostic methods that are as reliable and accurate as possible. It has now been found that such an improvement in reliability and accuracy can be achieved by the methods disclosed in the present invention, in which the assay of STR markers is undertaken using a co-amplification approach based on at least three simultaneous amplification assays.

The present invention relates to a diagnostic method for the detection of chromosomal abnormalities in a developing fetus and/or a new-born individual, or subsequently during adult growth. The method is based upon analysis of samples using the quantitative-fluorescent polymerase chain reaction (QF-PCR) to quantify sample DNA. Briefly, the method comprises the steps of: (a) simultaneously amplifying a plurality of chromosome-specific short tandem repeat (STR) markers to form an amplification product mixture comprising copies of the STR markers; (b) separating the amplified chromosome-specific STR markers from the amplified product mixture according to size; (c) determining the relative concentrations of the amplified products corresponding to the chromosome-specific STR markers, and (d) correlating the relative concentration of each amplified STR markers with the control chromosomes, followed with a confirmatory test.

At the gestational age of 15 weeks, approximately 50 to 100 ng of DNA can be obtained from approximate 1 ml of amniotic fluid. After DNA extraction, analytic results can be obtained within 4-5 hours following PCR amplification and fragment analysis with an auto sequencer. Overall, the results may be available within 8 hours after the arrival of amniotic fluid or CVS samples in a laboratory. QF-PCR is considered as one of the ideal methods that allow rapid, simple, and reliable detection of genomic markers for prenatal diagnosis. This approach has been designed to specifically identify genomic aberrations, such as chromosomes 13, 18, 21, X and Y aneuploidy as well as 22q11.2 deletions. Furthermore, the same technology may also be utilized for “non-invasive” prenatal analysis of DNA extracted from fetal cells existing in maternal blood samples, which eliminates the invasive procedure-related risks to the fetus.

### MODES FOR CARRYING OUT THE INVENTION

Before the present assays used to detect and confirm genomic aberrations is disclosed and described, it is to be understood that this invention is not limited to the particular configurations, process steps, and materials disclosed herein as such configurations, process steps, and materials may vary somewhat. It is also to be understood that the terminology employed herein is used for the purpose of describing particular embodiments only and is not intended to be limiting since the scope of the present invention will be limited only by the appended claims and equivalents thereof.

It must be noted that, as used in this specification and the appended claims, the singular forms “a,” “an,” and “the” include plural referents unless the context clearly dictates otherwise. Thus, for example, reference “a STR marker” includes reference to two or more such STR markers.

In describing and claiming the present invention, the following terminology will be used in accordance with the definitions set out below.

The condition of “aneuploidy” (or heteroploidy) refers to the condition of a cell nucleus having more than or less than an integral multiple of the typical haploid chromosome number. The term includes the conditions of monosomy where one chromosome of a chromosome pair is missing and trisomy where an additional copy is present. In some rare cases it is also possible for an individual to have two or more extra chromosomes.

The normal diploid number of chromosomes in humans is 46. Individuals with chromosome counts that are not multiples of the normal haploid number (23) are said to be aneuploid. A fetus can receive higher multiples of the haploid number of chromosomes to give 69 (3-times) or 92 (4-times) chromosomes. Such triploid or tetraploid fetuses normally miscarry early during pregnancy.

As used herein, the term “PCR primers” refers to primers complementary to sequences adjacent to an STR to be amplified. The PCR primers may be suitably in the range of from 15 to 35 nucleotides long, or in the range of from 10 to 50 nucleotides long, up to about 100 to 400 nucleotides of the STR to be amplified.

According to a first aspect of the present invention there is provided a method for detecting aneuploidy of a chromosome, the method comprising the steps of:
(a) simultaneously amplifying a plurality of chromosome-specific short tandem repeat (STR) markers to form an amplification product mixture comprising copies of one or more STR markers;
(b) separating the amplified chromosome-specific STR markers from the amplified product mixture according to size;
(c) determining the relative concentrations of the amplified products corresponding to the one or more chromosome-specific STR markers, and
(d) correlating the relative concentration of each amplified STR with the presence or absence of aneuploidy of the chromosomes.

As noted above, the methods of the present invention have particular importance with regard to the diagnosis of aneuploidy in a fetus. It should be noted that the methods are applicable of the determination of the chromosomal complement in any cell, i.e., all somatic and germ cells in an individual. In terms of a developing fetus, the methods may be practiced on any cell, from the one-cell zygote stage, through, the various embryonic stages to the development of the fetus. Additionally, there are sources of fetal DNA present in maternal plasma. In these samples, the DNA is free from the cell.

Examples of human disease conditions caused by aneuploidy include, but are not limited to, Down Syndrome (trisomy 21) i.e. three copies of chromosome 21, Edwards Syndrome (trisomy 18), Patau Syndrome (trisomy 13), Turner Syndrome (monosomy X) i.e. only one X chromosome in females, Kleinfelter Syndrome (XXY) in males, Triple X Syndrome (XXX) and other conditions such as (XYY).

The number of STR markers (STR regions) assayed according to the methods of the present invention, comprises a plurality of STR markers, preferably at least three STR markers per chromosome. Additional STR markers can be considered and independently included, so the number can be six, seven, eight, nine or ten, or more independently in total in each separate assay. Each assay can therefore contain a different number of markers.

In general, the STR marker DNA to be analyzed is amplified by polymerase chain reaction (PCR), a technique which is now standard in molecular biology laboratories. Primers for PCR amplification may be readily synthesized by standard techniques, for example by solid phase synthesis via phosphoramidite chemistry.

Chromosome-specific STR markers can be selected by choosing synthesizing primers that hybridize to adjacent unique sequence regions. The unique sequence regions will ensure that only the STR specific for the desired chromosome will be amplified. Appropriate STRs may be identified from publicly available DNA sequence data bases, such as GeneBank, or can be identified from libraries of chromosome-specific DNA libraries using the method described by Edwards et al, Am J Hum. Genet 49 746-756 (1991). STR markers can be obtained from the genome database (www.gdb.org) or the publication of the human genome (Science 291 1304-1351 (2001); Nature 409 813-958 (2001)) can be inspected. The STR markers can be selected from any desired locus on a chromosome that has the necessary heterozygosity. For disease conditions known to be associated with one of chromosomes 13, 18, 21, X or Y, the STR markers can be selected from loci on the chromosome as appropriate.

Several factors can affect the selection of primers for amplification, for example the relative stability of the primers when bound to target DNA which largely depends on their relative GC content, the presence or absence of secondary structures in the target DNA, the relative length of the primers (Rychlik et al, Nucleic Acids Research, 17 8543-8551 (1989); Lowe et al, Nucleic Acids Research, 18 1757-1761 (1990); Hillier et al, PCR Methods and Applications, 1 124-128 (1991)). Where a PCR machine is used, the STRs can be amplified by 20 to 35 PCR cycles, suitably by 25 to 30 PCR cycles.

In the methods of the present invention, it may be preferable for the amplification products (i.e. the copies of STR DNA produced in the amplification step) to be labeled to facilitate their quantification after separation. A variety of different labeling approaches are suitable for use with the present invention, including the direct or indirect attachment of radioactive labels, fluorescent labels, electron dense labels. There are several means available for derived oligonucleotides with reactive functional groups that permit the addition of a label. For example, several approaches are available for biotinylating a PCR primer so that fluorescent, enzymatic, or electron density labels can be attached via avidin (Broken et al, Nucleic Acids Research 5 363-384 (1978)), or by biotinylation of the 5′ termini of oligonucleotides via an aminoalkylphosphoramide linker arm (Chollet et al, Nucleic Acids Research 13 1529-1541 (1985)). Several methods are also available for synthesizing amino-derived oligonucleotides which are readily labeled by fluorescent or other types of compounds derived by amino-reactive groups, such as isothiocyanate, N-hydroxysuccinimide, (Connolly, Nucleic Acids Research 15.3131-3139 (1987); Gibson et al, Nucleic Acids Research 15 6455-6467 (1987); U.S. Pat. No. 4,605,735). Methods are also available for synthesizing sulfhydryl-derivatised oligonucleotides that can be reacted with thiol-specific labels, (U.S. Pat. No. 4,757,141; Connolly, Nucleic Acids Research 13.4485-4502 (1985); Spoat et al, Nucleic Acids Research, 15 4837-4848 10(1987). A comprehensive review of methodologies for labeling DNA fragments is provided by Matthews et al, Anal. Biochem., 169 1-25 (1988).

Amplified STR DNA can be labeled fluorescently by linking a fluorescent molecule to one or more primers. Preferably, copies of different STRs are labeled with different fluorescent labels to facilitate quantification. Preferred fluorescent labels include, fluorescein and derivatives thereof, tetramethylrhodamine, rhodamine X, Texas Red, and other related compounds. Most preferably, when plurality fluorescent dyes are employed they are spectrally resolvable, as taught by Fung et al., (cited above). Briefly, as used herein “spectrally resolvable” fluorescent dyes are those with quantum yields, emission bandwidths, and emission maxima that permit electrophoretically separated polynucleotides labelled thereby to be readily detected despite substantial overlap of the concentration bands of the separated polynucleotides.

PCR primers of the invention can also be radioactively labelled with phosphorous-32 using standard protocols. Separation of the amplified STRs from a sample by size fractionation may be accomplished in a variety of ways, including by filtration, high performance liquid chromatography, electrophoresis, affinity collection. The amplified STRs can be separated from the amplified product mixture by gel electrophoresis or capillary electrophoresis. Alternatively, the amplified STRs can be fluorescently labeled and separated by gel electrophoresis or capillary electrophoresis.

Chromosomal DNA of an individual who is being tested for aneuploidy is obtained from a cell sample from that individual or from a cell-free source, such as maternal blood plasma (Prenatal Diagnosis 20 795-798 (2000)). Cell samples can be obtained from a variety of tissues depending on the age and condition of the individual. Samples (cell or cell-free) may be obtained from peripheral blood using standard techniques. Preferably, in fetal testing, a sample is obtained by amniocentesis, chorionic villi sampling, or a sample of maternal blood plasma. Preferably, DNA is extracted from the sample using standard procedures. Cell samples for fetal testing can also be obtained from maternal peripheral blood using fluorescence-activated cell sorting (Iverson et al., Prenatal Diagnosis, 9.31-48 (1981)).

The correlation of the relative concentration of each amplified STR marker with the presence or absence of aneuploidy can be undertaken by any generally convenient means. The ratios of amplified STR marker products obtained in the method are analyzed and the diagnosis of the condition of the chromosomes in the sample can be made. Consistent results from at least two markers of the chromosome being assayed (with no opposing results) are required for an accurate diagnosis according to the method of the present invention.

In practice, diagnosis is only accurate when markers are selected carefully which produce smaller variation between allele size to avoid preferential amplification but maintain a high (over 70%) heterozygosity. A preferred embodiment of the present invention, includes a method as described above in which the STR markers have a high heterozygosity, of at least 70%, up to 75%, 80%, 85%, 90%, 95% or 100%.

In certain circumstances it may be convenient to arrange for an additional amplification assay to be carried out. For example, where a particular selection of markers has not yielded a clear result and only one STR marker shows a double-peak indicative of heterozygosity, then a further assay of additional STR markers can be used to confirm a diagnosis. Such additional markers can be used in combination, for example up to 3, or more, to provide additional data.

The STR markers that can be used in accordance with methods of the present invention, include but are not limited to: D13S800, D13S797, D13S796, D13S1493, D13S801, D13S325, D13S317, D18S877, D18S847, D18S379, D18S547, D18S976, D18S851, D18S1371, D21S1994, D21S1809, D21S1432, D21S2052, D21S1446, D21S1411, D21S1435, D21S1442, DXS7132, DXS981, DXS1187, AMXY, DXS2501, DYS19, DYS392, DXS7423, DXYS267, D22S311, D22S446, D22S944, D22S689, D22S685, D22S420, D22S264, etc.

The DNA to be analyzed can be obtained from any generally suitable cell, fluid or tissue source. In prenatal diagnosis, cells may be obtained from the developing fetus directly by tissue biopsy, or a sample of amniotic fluid or following chorionic villus sampling. In new born babies, children or adults, the sample can be obtained from any convenient tissue source, including, for example, blood or buccal swabs.

The results of the amplification procedure may be analyzed using a DNA sequencer. The relative amounts of amplification product can be measured according to the label used, e.g. fluorescent dye or radioactive label. When the results are displayed graphically, the area under the peak on the output from the sequence analyzer can be used to measure the amount of amplification product present for each DNA marker.

In reaching a diagnosis of trisomy, the ratios of the peaks obtained for each amplification product are compared. Methods in accordance with the present invention permit a diagnosis of a normal chromosomal complement with a peak ratio in the range 1:1 to 1.4:1 for a particular STR marker. A diagnosis of di-allelic trisomy (diplozygous trisomy) can be made when the peak ratio is above 1.6:1. The identification of these ratio values is important as false negative results are avoided.

In a preferred embodiment of the invention, there is a method provided as described in accordance with the first aspect of the invention, in which at least three simultaneous assays (or mulitplex mixes) each comprise independently at least six different STR markers (at least two markers for each chromosome being assayed for), and in which a peak value ratio of amplification product of a STR marker of 1:1 to 1.4:1 is diagnostic of a normal complement of chromosomes and a peak value ratio of 1.6:1 or above is diagnostic of di-allelic trisomy.

The XY assay is a little deferent from the other assays. The AMXY marker amplifies non-polymorphic sequences on the X(102bp) and Y(108bp) chromosomes and can be used to determine the presence or absence of a Y chromosome and gives the relative amount of X and Y products. All Y-specific markers will give a single peak in normal males and will not amplify in normal females and patients with Turner syndrome. All the X-specific markers will normally yield two peaks in normal females and give a single peak in patients with Turner syndrome. In a very infrequent case of uniparental disomy of chromosome X in a female, a result with no amplification for Y-specific markers and homozygous amplification for all X-specific markers will occur, which shows the same findings as in patients with Turner syndrome. In this situation, the present approach cannot provide a definite diagnosis for Turner syndrome, which demonstrates the limitations in this matter.

According to a second aspect of the present invention, there is a provided kit of parts comprising at least three multiplexes of labeled primers for carrying out the method of the present invention as described above. Suitably such kits can include at least 3 sets of labeled primers for the STR markers to be amplified, polymerase buffer solution in which a DNA polymerase can extend the primers in the presence of DNA polymerase, and deoxynucleoside triphosphates. The labeled primers may include fluorescent labels and the DNA polymerase may be Taq DNA polymerase. The fluorescent labels include, but are not limited to, fluorescein, rhodamine, and derivatives thereof, including carboxyfluorescein, 4,7-dichlorofluoresceins, tetramethylrhodamine, rhodamine X, or derivatives thereof.

These kits include but are not limited to CYT-T, CYT-D, CYT-13, CYT-18, CYT-21, CYT-22, CYT-XY. Information including marker name, marker location, allele size range, observed heterozygosity, repeat type and marker dye color for these kits is enumerated below in Table 1.

CYT-T is used to initially detect the three most common autosomal trisomies: trisomy 13 (Patau syndrome), trisomy 18 (Edwards syndrome) and trisomy 21 (Down syndrome); CYT-D is used to initially detect sex chromosome abnormalities including monosomy X (Turner Syndrome), XXY (Kleinfelter Syndrome), XXX (Triple X Syndrome), XYY syndrome as well as 22q11.2 deletion syndromes. CYT-13, CYT-18, CYT-21, CYT-XY and CYT-22 are used to further confirm the abnormalities as identified with the above initial detection kits.

TABLE 1
Representative markers employed

| | | | | | |
|---|---|---|---|---|---|
| Marker | Location | Allele size range | Het. | Repeat | Primer dye color |

Markers in CYT-T

| | | | | | |
|---|---|---|---|---|---|
| D13S800 | 13q22.1 | 284-324 | 0.75 | Tetra | blue |
| D21S1994 | 21q21.1 | 235-264 | 0.71 | Tetra | blue |
| D13S797 | 13q33.2 | 176-208 | 0.67 | Tetra | blue |
| D18S877 | 18q12.1 | 103-155 | 0.68 | Tetra | blue |
| D18S379 | 18q21.33 | 263-323 | 0.89 | Tetra | green |
| D18S847 | 18q12.1 | 204-236 | 0.76 | Tetra | green |
| D13S796 | 13q33.3 | 127-175 | 0.8 | Tetra | green |
| D21S1809 | 21q22.2 | 192-224 | 0.7 | Tetra | yellow |
| D21S2052 | 21q21.3 | 113-165 | 0.86 | tetra | yellow |

Markers in CYT-D

| | | | | | |
|---|---|---|---|---|---|
| DXS7132 | Xq11.1 | 276-308 | | Tetra | blue |
| D22S446 | 22q11.21 | 198-232 | 0.81 | Di | blue |
| D22S944 | 22q11.21 | 158-178 | | Di | blue |
| AMXY | Yp11.2, Xp22 | 102 for X, 108 for Y | | — | blue |
| DXS2501 | XP11.3 | 255-299 | 0.76 | Tetra | green |
| D22S689 | 22q12.1 | 194-238 | 0.76 | Tetra | green |
| D22S420 | 22q11.1 | 139-171 | 0.77 | Di | green |
| DYS392 | Yq11.222 | 254- | 0.56 | Tri | yellow |
| D22S264 | 22q11.21 | 190-210 | | Di | yellow |
| DXYS267 | Xq21.31/yp11.2 | 123- | 0.87 | Tetra | yellow |

Markers in CYT-13

| | | | | | |
|---|---|---|---|---|---|
| D13S1493 | 13q13.2 | 210-270 | 0.77 | Tetra | blue |
| D13S801 | 13q21.31 | 170-194 | | Tetra | blue |
| D13S325 | 13q14.11 | 195-251 | 0.8 | Tetra | green |
| D13S796 | 13q33.3 | 127-175 | 0.8 | Tetra | green |
| D13S317 | 13q31.1 | 172-208 | 0.79 | Tetra | yellow |

Markers in CYT-18

| | | | | | |
|---|---|---|---|---|---|
| D18S547 | 18q21.1 | 248- | | Tetra | blue |
| D18S976 | 18p11.31 | 160-200 | 0.86 | Tetra | blue |
| D18S851 | 18q21.2 | 237-285 | 0.73 | Tetra | green |
| D18S847 | 18q12.1 | 204-236 | 0.76 | Tetra | green |
| D18S1371 | 18q22.3 | 133-162 | 0.7 | Tetra | green |

Markers in CYT-21

| | | | | | |
|---|---|---|---|---|---|
| D21S1994 | 21q21.1 | 235-264 | 0.71 | Tetra | blue |
| D21S1446 | 21q22.3 | 185-233 | 0.69 | Tetra | blue |
| D21S1411 | 21q22.3 | 239-324 | 0.89 | Tetra | green |
| D21S1435 | 21q21.3 | 152-210 | 0.75, (0.81) | Tetra | green |
| D21S1442 | 21q21.3 | 237-269 | 0.86 | tetra | yellow |
| D21S1809 | 21q22.2 | 192-224 | 0.7 | Tetra | yellow |
| D21S1432 | 21q21.1 | 124-160 | 0.69 | tetra | yellow |

Markers in CYT-XY

| | | | | | |
|---|---|---|---|---|---|
| DXS7132 | Xq11.1 | 276-308 | 0.73 | Tetra | blue |
| DXS981 | Xq13.1 | 230-260 | 0.86 | Tetra | blue |
| DXS1187 | Xq26.2 | 130-170 | 0.72 | Tetra | blue |
| AMXY | Yp11.2, Xp22 | 102 for X, 108 for Y | | — | blue |
| DXS2501 | XP11.3 | 255-299 | 0.76 | Tetra | green |
| DYS19 | yp11.2 | 186-202 | 0.66 | Tetra and di | green |
| DYS392 | Yq11.222 | 254- | 0.56 | Tri | yellow |
| DXS7423 | xq28 | 161-181 | 0.73 | Tetra | yellow |
| DXYS267 | Xq21.31/yp11.31 | 123- | 0.87 | Tetra | yellow |

Markers in CYT-22

| | | | | | |
|---|---|---|---|---|---|
| D22S311 | 22q11.21 | 262-276 | 0.806 | Di | blue |
| D22S446 | 22q11.21 | 198-232 | 0.81 | Di | blue |
| D22S944 | 22q11.21 | 158-178 | | Di | blue |
| D22S689 | 22q12.1 | 194-238 | 0.76 | Tetra | green |
| D22S685 | 22q.12.3 | 164-216 | 0.79 | Tetra | green |
| D22S420 | 22q11.1 | 139-171 | 0.77 | Di | green |
| D22S264 | 22q11.21 | 190-210 | | Di | yellow |

These kits are offered in a reaction mix form, containing 10 mM Tris-HCL, pH 8.3, 50 mM KCL, 1.0-5.0 mM MgCL₂, 100-500 uM deoxynucleotide triphosphates (dNTP), 0.5-4 u/ul DNA polymerase, 0.01-2.0 uM primers and any other elements necessary for DNA amplification except DNA template. Blue, green, or yellow fluorescent dyes were used to label different primers in order to separate the amplified chromosome-specific STR markers that have similar length. The fluorescent labels include, but are not limited to FAM, HEX, TAMRA.

The reaction mix was dispensed into 10-20 ul. 0.5-10 ng of genome DNA in a volume of about 2.0 ul was added into each portion. The thermal cycler was set up as follows: 5 to 15 minutes at 95° C. for 1 cycle; 30 seconds to 1 minute at 94° C., 30 seconds to 1 minute and 30 seconds at 60° C., 30 seconds to 1 minute and 30 seconds at 72° C. for 25 to 30 cycles; 30 minutes to 1 hour at 60 to 72° C. for 1 cycle.

On completion of the amplification program the samples could be stored at room temperature overnight or at 2-8° C. for up to 7 days before analysis by capillary electrophoresis. Optimal results were obtained by using an ABI 3100 genetic analyzer or its upgraded editions.

According to a third aspect of the invention there is the use of STR marker 21-32S (informal designation) provided as a marker for the diagnosis of aneuploidy. The method may be as described above in relation to the first aspect of the invention, or alternatively, the method may be any generally suitable diagnostic test.

Unlike FISH, the methods of the present invention are feasible on very small volumes of amniotic fluid (0.3 to 1 ml), which does not then compromise any cell culture requirements. The PCR methodology amplifies DNA from cells and therefore does not rely on the cells being alive or intact. This allows the technique to be used on samples taken at both earlier (12 weeks) or later gestations (34 weeks), when samples are lacking an abundance of live cells, without affecting its reliability. QF-PCR can be easily scaled up to cope with large numbers of samples (e.g. 240 samples per 24 hours per 3700 ABI DNA Sequencer).

Concerns about maternal cell contamination in cell cultures have been alleviated simply by comparing the DNA profiles from the maternal DNA obtained from a mouthwash sample to the profile from the amniotic fluid DNA.

In a preferred embodiment of the invention, there is provided a method for detecting aneuploidy of a chromosome comprising the following steps:
(1) preparing sample(s) of amniotic fluid for analysis;
(2) selecting appropriate chromosome-specific short tandem repeat (STR) markers for use in at least 3 simultaneous multiplex reactions, including preparation of negative controls with no template DNA;
(3) labeling the forward or reverse primer from each pair of markers used with appropriate label (e.g. fluorescent dye, radioactive label);
(4) preparing sample mixtures for polymerase chain reaction (PCR);
(5) amplifying the DNA sequences in samples using PCR;
(6) separating the amplified DNA samples, e.g. by electrophoresis;
(7) quantifying the DNA representing each allele amplified for a specific marker used in the 3 simultaneous multiplex reactions;
(8) analyzing ratios of peak area for each allele amplified and determination of the chromosome status of the fetus.

The samples for analysis may be frozen, or if routine culture is to be performed in addition then samples are at room temperature.

The extraction of DNA from the cells may be performed by any convenient means. The cells may be resuspended and a 1.0 ml aliquot centrifuged in a microfuge tube. The pellet of cells may then be resuspended in a suitable medium such as phosphate buffered saline to wash the cells. The pellet may then be resuspended and incubated at an appropriate temperature of at least 50° C., preferably 56° C. and no more than 60° C. The DNA thus obtained is denatured by heating at 100° C. and then centrifuged.

The QF-PCR may be performed as follows. For each sample or control, three sets of tubes are prepared each containing one of the three different multiplex mixes of probe/primer sets as desired. Supernatant containing DNA from the cell sample is then pipetted into each set of tubes, including controls. The sample tubes thus prepared are subjected to PCR using a convenient apparatus.

At the end of the PCR, the samples are separated by gel or capillary electrophoresis using conventional fluorescent DNA analyzers. Identification and quantification of DNA products can be performed using any convenient method. The DNA fragment size, chromosomal origin and quantification can then be determined using any generally convenient means. Markers are identified for each chromosome pair and are classified by comparison to results from known samples.

In such a method, markers producing three peaks with an approximate peak area ratio of 1:1:1 (i.e. below 1.4) are considered consistent with trisomy. Heterozygous markers producing two peaks with a DNA ratio below 1.4 are considered to be consistent with euploidy and a ratio above 1.6 consistent with trisomy. Any ratio between 1.4 and 1.6 is considered to be inconclusive. PCR reactions producing inconclusive ratios may be repeated to clarify the results. In cases where only one marker from a single chromosome is found to be heterozygous, an extra multiplex system comprised of at least two different DNA markers per chromosome can be used. Positive and consistent results from at least two informative markers for each chromosome are required before a conclusion is drawn.

The following example will enable those skilled in the art to more clearly understand how to practice the present invention. It is to be understood that, while the invention has been described in conjunction with the preferred specific embodiments thereof, that which follows is intended to illustrate and not limit the scope of the invention. Other aspects of the invention will be apparent to those skilled in the art to which the invention pertains.

### EXAMPLE 1

### Identification of Chromosomes 13, 18, 21, X, and Y Aneuploidy as Well as 22q11.2 Deletion for Prenatal Diagnosis

This example illustrates the method of this invention using QF-PCR and capillary electrophoresis (CE) techniques to detect genomic aberrations, such as chromosome 13, 18, 21, X and Y aneuploidy as well as 22q11.2 deletion. By PCR amplification, different fluorescent labeled primers target highly polymorphic areas of the genomic DNA sequence i.e. STRs, especially tetra-nucleotide repeats, which are located on the chromosomes of interest (such as chromosomes 13, 18, 21, X, Y and 22q11.2). Each targeted STR is specific to the chromosome on which it is located. The method uses the primers to co-amplify a panel of, for example, 12 micro-satellite loci of tetranucleotide repeats of human genomic DNA, such as D13S1493, D13S317, D13S796, D18S976 D18S974, D18S541, D21S1442, D21S1435, D21S1809, D22S446, D22S689 and AMXY. All of these tetranucleotide repeat markers can be obtained from the genome database (www.gdb.org). The following is the list for the detailed sequences of the representative primers used.

After simultaneously amplifying at least three microsatellite loci of the panel from DNA of a sample in a multiplex amplification reaction to produce amplified DNA fragments; the sizes of the amplified DNA fragments are measured. Combined with the heterozygosity of at least >0.75 of selected informative STRs, copy numbers can be easily determined. In addition, by optimizing PCR parameters, fluorescent intensity is also used for quantitative analysis. For example, a normal diploid specimen shows two peaks in a 1:1 ratio of a chromosome specific STR. For the same STR, an observation of an extra peak (three peak pattern) in a 1:1:1 ratio or a two peak pattern in a 2:1 ratio may suggest the presence of an additional sequence, representing the additional chromosome (trisomy). Negative controls with no template DNA are included for each of the multiplexes on every run. Follow-up confirmatory tests are recommended to further confirm any initial positive testing results before reporting out.

### EXAMPLE 2

### Confirmation of Chromosome 13 Aneuploidy as Identified by FISH or QF-PCR Methods in Prenatal Diagnosis

To confirm the presence of chromosome 13 aneuploidy in prenatal diagnosis as revealed by FISH or QF-PCR techniques, additional specifically designed STRs on chromosome 13 are utilized for further analysis. The confirmatory test examines, for example, 7 different microsatellite loci across chromosome 13 and 2 commonly used sex-linked markers; so quantitative analysis can be performed. The method uses the primers to co-amplify a set of 9 microsatellite loci of human genomic DNA, such as D13S1493, D13S325, D13S801, D13S800, D13S317, D13S797, D13S796, AMXY, and DXS8377. At least three microsatellite loci of the set from the DNA sample are co-amplified in a multiplex amplification reaction to generate amplified DNA fragments. All of the tetranucleotide repeat markers can be obtained from the genome database (www.gdb.org). The sizes of the amplified DNA fragments are then measured. It is expected to see either all of the 7 markers showing trisomy or at least 2 markers showing trisomy to indicate the presence of complete or partial trisomy 13, respectively.

### EXAMPLE 3

### Confirmation of Chromosome 18 Aneuploidy as Detected by FISH or QF-PCR Approach in Prenatal Diagnosis

To verify the presence of chromosome 18 aneuploidy in prenatal diagnosis as revealed by FISH or QF-PCR techniques, additional specifically designed STRs on chromosome 18 are employed for further analysis. The confirmatory test examines, for example, 7 different microsatellite loci across chromosome 18 and 2 commonly used sex-linked markers; so quantitative analysis can be performed. The method uses the primers to co-amplify a set of 9 microsatellite loci of human genomic DNA, such as D18S1976, D18S542, D18S877, D18S847, D18S974, D18S1270, D18S541, AMXY and DXS8377. At least three microsatellite loci of the set from the DNA sample are co-amplified in a multiplex amplification reaction to generate amplified DNA fragments. All of the tetranucleotide repeat markers can be obtained from the genome database (www.gdb.org). The sizes of the amplified DNA fragments are then measured. It is expected to see either all of the 7 markers showing trisomy or at least 2 markers showing trisomy to indicate the presence of complete or partial trisomy 18, respectively.

### EXAMPLE 4

### Confirmation of Chromosome 21 Aneuploidy as Detected by FISH or QF-PCR Methods in Prenatal Diagnosis

To confirm the presence of chromosome 21 aneuploidy in prenatal diagnosis as revealed by FISH or QF-PCR techniques, additional specifically designed STRs on chromosome 21 are utilized for further analysis. The confirmatory test examines, for example, 6 different microsatellite loci across chromosome 21 and 2 commonly used sex-linked markers; so quantitative analysis can be performed. The method uses the primers to co-amplify a set of 8 microsatellite loci of human genomic DNA, such as D21S1442, D21S1437, D21S1435, D21S1270, D21S1809, D21S1446, AMXY and DXS8377. At least three microsatellite loci of the set from the DNA sample are co-amplified in a multiplex amplification reaction to generate amplified DNA fragments. All of the tetranucleotide repeat markers can be obtained from the genome database (www.gdb.org). The sizes of the amplified DNA fragments are then measured. It is expected to see either all of the 6 markers showing trisomy or at least 2 markers showing trisomy to indicate the presence of complete or partial trisomy 21, respectively.

### EXAMPLE 5

### Confirmation of 22q11.2 Deletion as detected by FISH or QF-PCR Methods in Prenatal Diagnosis

To confirm the presence of 22q11.2 deletion in prenatal diagnosis as revealed by FISH or QF-PCR techniques, additional specifically designed STRs on chromosome 22 are utilized for further analysis. The confirmatory test examines, for example, 4 different microsatellite loci covering the specific region of 22q11.2 and 2 commonly used sex-linked markers; so quantitative analysis can be performed. The method uses the primers to co-amplify a set of 6 microsatellite loci of human genomic DNA, such as D22S420, D22S446, D22S689, D22S685, AMXY and DXS8377. At least three microsatellite loci of the set from the DNA sample are co-amplified in a multiplex amplification reaction to generate amplified DNA fragments. All of the tetranucleotide repeat markers can be obtained from the genome database (www.gdb.org). The sizes of the amplified DNA fragments are then measured. It is expected to see either all of the 6 markers showing deletion or at least 2 markers showing deletion to indicate the 22q11.2 deletion, respectively.

### EXAMPLE 6

### Confirmation of Chromosomes X and Y Aneuploidy as Detected by FISH or QF-PCR Methods in Prenatal Diagnosis

To confirm the presence of chromosome X and Y aneuploidy in prenatal diagnosis as revealed by FISH or QF-PCR techniques, additional specifically designed STRs on chromosomes X and Y are utilized for further analysis. The confirmatory test examines, for example, 3 different microsatellite loci so the copy numbers can be determined by comparing fluorescent intensity between the sample and control DNA provided. The method uses the primers to co-amplify a set of 3 microsatellite loci of human genomic DNA, such as AMXY, DXS7432 and DXS8377. All of the tetranucleotide repeat markers can be obtained from the genome database (www.gdb.org). The sizes of the amplified DNA fragments are then measured. It is expected to see either all of the 3 markers showing aneuploidy \or at least 2 markers showing aneuploidy to indicate chromosome X and Y aneuploidy, respectively.

It is to be understood that the above-described embodiments are only illustrative of application of the principles of the present invention. Numerous modifications and alternative embodiments can be derived without departing from the spirit and scope of the present invention and the appended claims are intended to cover such modifications and arrangements. Thus, while the present invention has been shown in the drawings and fully described above with particularity and detail in connection with what is presently deemed to be the most practical and preferred embodiment(s) of the invention, it will be apparent to those of ordinary skill in the art that numerous modifications can be made without departing from the principles and concepts of the invention as set forth in the claims.

1. A method of detecting genomic aberrations, comprising the steps of: (a) providing a panel of primers for amplifying chromosome-specific short tandem repeat (STR) markers selected from the group consisting of D13S 1493, D13S317, D13S796, D18S976, D18S974, D18S541, D21S1442, D21S1435, D21S1809, D22S446, D22S689 and AMXY; (b) simultaneously amplifying at least three said STR markers from a sample in a multiplex amplification reaction to produce an amplified DNA fragments mixture comprising copies of the STR markers; (c) separating the amplified chromosome-specific STR markers from the amplified product mixture according to size; (c) determining the relative concentrations of the amplified products corresponding to the chromosome-specific STR markers, and (d) correlating the relative concentration of each amplified STR with the presence or absence of aneuploidy of the chromosome, followed by a confirming test.

2. The method according to claim 1, wherein said genomic aberration is a member selected from the group consisting of chromosomes 13, 18, 21, X, Y aneuploidy, and 22q11.2 deletion.

3. The method according to claim 1, wherein the confirming test for chromosome 13 aneuploidy, comprises the steps of: (a) providing a panel of primers for amplifying chromosome-specific short tandem repeat (STR) markers selected from the group consisting of D13S1493, D13S325, D13S801, D13S800, D13S317, D13S797, D13S796, AMXY and DXS8377; (b) simultaneously amplifying at least three said STR markers of a sample in a multiplex amplification reaction to produce an amplified DNA fragments mixture comprising copies of the STR markers; (c) separating the amplified chromosome-specific STR markers from the amplified product mixture according to size; (d) determining the relative concentrations of the amplified products corresponding to the chromosome-specific STR markers.

4. The method according to claim 1, wherein the confirming test for chromosome 18 aneuploidy, comprises the steps of: (a) providing a panel of primers for amplifying chromosome-specific short tandem repeat (STR) markers selected from the group consisting of D18S1976, D18S542, D18S877, D18S847, D18S974, D18S1270, D18S541, AMXY and DXS8377; (b) simultaneously amplifying at least three said STR markers of a sample in a multiplex amplification reaction to produce an amplified DNA fragments mixture comprising copies of the STR markers; (c) separating the amplified chromosome-specific STR markers from the amplified product mixture according to size; (c) determining the relative concentrations of the amplified products corresponding to the chromosome-specific STR markers.

5. The method according to claim 1, wherein the confirming test for chromosome 21 aneuploidy, comprises the steps of: (a) providing a panel of primers for amplifying chromosome-specific short tandem repeat (STR) markers selected from the group consisting of D21S1442, D21S1437, D21S1435, D21S1270, D21S1809, D21S1446, AMXY and DXS8377; (b) simultaneously amplifying at least three said STR markers of a sample in a multiplex amplification reaction to produce an amplified DNA fragments mixture comprising copies of the STR markers; (c) separating the amplified chromosome-specific STR markers from the amplified product mixture according to size; (d) determining the relative concentrations of the amplified products corresponding to the chromosome-specific STR markers.

6. The method according to claim 1, wherein the confirming test for chromosomes X and Y aneuploidy comprises the steps of: (a) providing a panel of primers for amplifying chromosome-specific short tandem repeat (STR) markers selected from the group consisting of AMXY, DXS7432 and DXS8377; (b) simultaneously amplifying at least three said STR markers of a sample in a multiplex amplification reaction to produce an amplified DNA fragments mixture comprising copies of the STR markers; (c) separating the amplified chromosome-specific STR markers from the amplified product mixture according to size; (c) determining the relative concentrations of the amplified products corresponding to the chromosome-specific STR markers.

7. The method according to claim 1, wherein the confirming test for chromosome 22q11.2 deletion comprises the steps of: (a) providing a panel of primers for amplifying chromosome-specific short tandem repeat (STR) markers selected from the group consisting of D22S420, D22S446, D22S689, D22S685, AMXY and DXS8377; (b) simultaneously amplifying at least three said STR markers of a sample in a multiplex amplification reaction to produce an amplified DNA fragments mixture comprising copies of the STR markers; (c) separating the amplified chromosome-specific STR markers from the amplified product mixture according to size; (c) determining the relative concentrations of the amplified products corresponding to the chromosome-specific STR markers.

8. The method according to claim 1, wherein the amplification of the STR markers is by means of a polymerase chain reaction.

9. The method according to claim 8 wherein the primer for the amplification reaction is labeled with a fluorescent label.

10. The method according to claim 8, the sample is a member selected from the group consisting of peripheral blood, amniotic fluid, cultured amniocytes, chorionic villi, or fetal cells existing in maternal blood.

11. The method according to claim 8, wherein said genomic aberration is a member selected from the group consisting of chromosomes 13, 18, 21, X, Y aneuploidy, and 22q11.2 deletion.

12. The method according to claim 8, wherein the confirming test for chromosome 13 aneuploidy, comprises the steps of: (a) providing a panel of primers for amplifying chromosome-specific short tandem repeat (STR) markers selected from the group consisting of D13S1493, D13S325, D13S801, D13S800, D13S317, D13S797, D13S796, AMXY and DXS8377; (b) simultaneously amplifying at least three said STR markers of a sample in a multiplex amplification reaction to produce an amplified DNA fragments mixture comprising copies of the STR markers; (c) separating the amplified chromosome-specific STR markers from the amplified product mixture according to size; (d) determining the relative concentrations of the amplified products corresponding to the chromosome-specific STR markers.

13. The method according to claim 8, wherein the confirming test for chromosome 18 aneuploidy, comprises the steps of: (a) providing a panel of primers for amplifying chromosome-specific short tandem repeat (STR) markers selected from the group consisting of D18S1976, D18S542, D18S877, D18S847, D18S974, D18S1270, D18S541, AMXY and DXS8377; (b) simultaneously amplifying at least three said STR markers of a sample in a multiplex amplification reaction to produce an amplified DNA fragments mixture comprising copies of the STR markers; (c) separating the amplified chromosome-specific STR markers from the amplified product mixture according to size; (c) determining the relative concentrations of the amplified products corresponding to the chromosome-specific STR markers.

14. The method according to claim 8, wherein the confirming test for chromosome 21 aneuploidy, comprises the steps of: (a) providing a panel of primers for amplifying chromosome-specific short tandem repeat (STR) markers selected from the group consisting of D21S1442, D21S1437, D21S1435, D21S1270, D21S1809, D21S1446, AMXY and DXS8377; (b) simultaneously amplifying at least three said STR markers of a sample in a multiplex amplification reaction to produce an amplified DNA fragments mixture comprising copies of the STR markers; (c) separating the amplified chromosome-specific STR markers from the amplified product mixture according to size; (d) determining the relative concentrations of the amplified products corresponding to the chromosome-specific STR markers.

15. The method according to claim 8, wherein the confirming test for chromosomes X and Y aneuploidy comprises the steps of: (a) providing a panel of primers for amplifying chromosome-specific short tandem repeat (STR) markers selected from the group consisting of AMXY, DXS7432 and DXS8377; (b) simultaneously amplifying at least three said STR markers of a sample in a multiplex amplification reaction to produce an amplified DNA fragments mixture comprising copies of the STR markers; (c) separating the amplified chromosome-specific STR markers from the amplified product mixture according to size; (c) determining the relative concentrations of the amplified products corresponding to the chromosome-specific STR markers.

16. The method according to claim 8, wherein the confirming test for chromosome 22q11.2 deletion comprises the steps of: (a) providing a panel of primers for amplifying chromosome-specific short tandem repeat (STR) markers selected from the group consisting of D22S420, D22S446, D22S689, D22S685, AMXY and DXS8377; (b) simultaneously amplifying at least three said STR markers of a sample in a multiplex amplification reaction to produce an amplified DNA fragments mixture comprising copies of the STR markers; (c) separating the amplified chromosome-specific STR markers from the amplified product mixture according to size; (c) determining the relative concentrations of the amplified products corresponding to the chromosome-specific STR markers.

17. The method according to claim 11 wherein the primer for the amplification reaction is labeled with a fluorescent label.

18. The method according to claim 11, the sample is a member selected from the group consisting of peripheral blood, amniotic fluid, cultured amniocytes, chorionic villi, or fetal cells existing in maternal blood.

19. The method according to claim 12 wherein the primer for the amplification reaction is labeled with a fluorescent label.

20. The method according to claim 12, the sample is a member selected from the group consisting of peripheral blood, amniotic fluid, cultured amniocytes, chorionic villi, or fetal cells existing in maternal blood.

21. The method according to claim 13 wherein the primer for the amplification reaction is labeled with a fluorescent label.

22. The method according to claim 13, the sample is a member selected from the group consisting of peripheral blood, amniotic fluid, cultured amniocytes, chorionic villi, or fetal cells existing in maternal blood.

23. The method according to claim 14, wherein the primer for the amplification reaction is labeled with a fluorescent label.

24. The method according to claim 14, the sample is a member selected from the group consisting of peripheral blood, amniotic fluid, cultured amniocytes, chorionic villi, or fetal cells existing in maternal blood.

25. The method according to claim 15 wherein the primer for the amplification reaction is labeled with a fluorescent label.

26. The method according to claim 15, the sample is a member selected from the group consisting of peripheral blood, amniotic fluid, cultured amniocytes, chorionic villi, or fetal cells existing in maternal blood.

27. The method according to claim 16, wherein the primer for the amplification reaction is labeled with a fluorescent label.

28. The method according to claim 16, the sample is a member selected from the group consisting of peripheral blood, amniotic fluid, cultured amniocytes, chorionic villi, or fetal cells existing in maternal blood.
